(19) United States
(12) Patent Application Publication
(10) Pub. No.: US 2019/0050535 A1
(43) Pub. Date: Feb. 14, 2019

(54) Method Of Classifying A Biological Sample

(71) Applicants: UNIVERSITE DE MONTPELLIER, Montpellier (FR); INSTITUT DE RECHERCHE POUR LE DEVELOPPEMENT, Marseille (FR); INSTITUT NATIONAL DE LA SANTE ET DE LA RECHERCHE MEDICALE, Paris (FR)

(72) Inventors: Jean-Christophe Avarre, Montferrier-sur-Lez (FR); Christelle Reynes, Montpellier (FR)

(73) Assignees: UNIVERSITE DE MONTPELLIER, Montpellier (FR); INSTITUT DE RECHERCHE POUR LE DEVELOPPEMENT, Marseille (FR); INSTITUT NATIONAL DE LA SANTE ET DE LA RECHERCHE MEDICALE, Paris (FR)

(21) Appl. No.: 16/071,857

(22) PCT Filed: Jan. 23, 2017
(86) PCT No.: PCT/EP2017/051327

§ 371 Date: Jul. 20, 2018

(30) Foreign Application Priority Data
Jan. 22, 2016 (FR) 1650527

(51) Int. Cl. G06F 19/24; C12Q 1/6816; G06F 19/20

(52) U.S. Cl. CPC G06F 19/24; G06F 19/20; C12Q 1/6816

(57) ABSTRACT

The present invention relates to a method for classifying a measurement biological sample, comprising: acquisition of at least one DNA melting curve of the measurement biological sample, called at least one measurement curve; and determination that the measurement biological sample belongs to a defined group among different possible groups, by analysis of descriptors originating from the at least one measurement curve, characterized in that the descriptors comprise one or more points of the first derivative of each measurement curve and/or comprise one or more points of the second derivative of each measurement curve and/or one or more points of each measurement curve and/or one or more percentiles of each measurement curve. The invention also relates to a device implementing this method.

### CROSS-REFERENCE TO RELATED APPLICATIONS

This application claims priority to PCT Application No. PCT/EP2017/051327, having a filing date of Jan. 23, 2017, based off of French application No. 1650527 having a filing date of Jan. 22, 2016, the entire contents of both of which are hereby incorporated by reference.

### TECHNICAL FIELD

The present invention relates to a method for classifying a biological sample, and an associated device.

Such a device allows a user to classify a biological sample among several possible groups. The field of the invention is that of biological classification.

### BACKGROUND

A known document, WO2013/166373, describes a method for determining the regulation status of the IL-6/STAT3 signalling pathway in a cell sample or in a subject. The regulation status of the IL-6/STAT3 signalling pathway in a cell sample or in a subject can be analysed on the basis of the level of expression of one or more among 16 genes of an expression signature. Biomarker expression is preferably determined by RT-PCR using SYBR Green methods, and expression data are analysed and compared with a control sample using the random forest method. Determination of the variables selected (in this case the 16 genes) is specific to the problem and must be carried out manually for each new problem.

Certain technical problems can arise for a method for classifying a biological sample in a group, in particular when the number of possible groups is large, for example:
How to improve the fineness of classification? and/or
How to be able to treat a sample that does not belong to any known group?
How to automate the method, without manual selection of variables?

The purpose of the present invention is to solve at least one of these problems.

### SUMMARY

This objective is achieved with a method for classifying a measurement biological sample, comprising:
acquisition of at least one DNA melting curve of the measurement biological sample, called at least one measurement curve (preferably acquisition of several DNA melting curves of the measurement biological sample, also called technical replicates), typically each measurement curve comprising different points, each point corresponding to a quantity that is proportional to or representative of a level or a quantity of denaturation of the DNA of the measurement sample as a function of a temperature,
determination that the measurement biological sample belongs to a defined group among different possible groups, by analysis of descriptors originating from the at least one measurement curve,
   characterized in that the descriptors comprise:
one or more (preferably several) point(s) of the first derivative of each measurement curve and/or comprise one or more (preferably several) point(s) of the second derivative of each measurement curve, and/or
one or more (preferably several) point(s) of the first derivative of each measurement curve and comprise one or more (preferably several) point(s) of the second derivative of each measurement curve, and/or
one or more point(s) of each measurement curve, and/or
one or more percentile(s) of each measurement curve.

The acquisition of at least one DNA melting curve of the measurement biological sample can comprise the acquisition of at least one melting curve of a result of a PCR obtained in the simultaneous presence of several primer pairs targeting several target DNA molecules, corresponding for example to several pathogens. This is then referred to as “multiplexing” conditions. This embodiment is useful for speeding up the search, for example, for several pathogens that are very rarely present together in one and the same biological sample. The rare cases for example of several pathogens present together in a sample are identified: typically, the melting curve has as many inflection points as pathogens present; different “defined groups” can then include the different combinations of the presence of these different pathogens.

The determination can comprise determination by a random forest method. The method according to the invention can comprise learning comprising:
acquisition of different DNA melting curves, called reference curves, starting from different reference biological samples belonging to different known initial groups determined before learning, then
determination of the descriptors from the reference curves, and then
construction of a forest by the random forest method, comprising construction of several trees by the random forest method, each tree comprising, at each node, the use of one of the descriptors associated with a threshold and making it possible to separate the reference curves into two subsets, each leaf of each tree only corresponding to a single group among the different possible groups.

Determination of the descriptors can comprise:
a preliminary determination of descriptors, and then
an elimination of certain redundant descriptors.

The elimination of certain descriptors can comprise, for each set of descriptors displaying in pairs a Pearson correlation coefficient greater than 0.95, retention of a single descriptor.

The method according to the invention can comprise:
after acquisition of different reference curves (and/or preferably before construction of the trees), identification of several reference curves corresponding to the same initial group, called ambiguous group, and having profiles separated into several subgroups, and
separation of this ambiguous group into several possible groups.

The method according to the invention can comprise:
after acquisition of different reference curves (and/or preferably after construction of the trees), identification of several reference curves corresponding to several initial groups, called merged groups, and having profiles combined in a single group, and
unification of these merged groups into a single possible group.

The method according to the invention can further comprise calculation of a confidence index of the step of determining that the measurement biological sample belongs to a defined group. The calculation of the confidence index can comprise:
calculation of a distribution of mean proximities between reference curves belonging to the defined group,
calculation of a mean proximity of the at least one measurement curve with the reference curves belonging to the defined group, and
calculation of a level of reference curves belonging to the defined group, and having a mean proximity to the other reference curves belonging to the defined group less than the mean proximity of the at least one measurement curve with the reference curves belonging to the defined group.

The method according to the invention can further comprise, after the step of determining that the measurement biological sample belongs to a defined group, a refusal to assign the measurement biological sample to any group whatever, as a function of the value of the confidence index.

According to yet another aspect of the invention, a device for classifying a measurement biological sample is proposed, comprising:
means arranged and/or programmed for acquiring at least one DNA melting curve of the measurement biological sample, called at least one measurement curve (preferably acquisition of several DNA melting curves of the measurement biological sample, also called technical replicates), typically each measurement curve preferably comprising different points, each point corresponding to a quantity that is proportional to or representative of a level or a quantity of denaturation of the DNA of the measurement sample as a function of a temperature,
means arranged and/or programmed for determining that the measurement biological sample belongs to a defined group among different possible groups, by analysis of descriptors originating from the at least one measurement curve,
   characterized in that the descriptors comprise:
one or more (preferably several) point(s) of the first derivative of each measurement curve and/or comprise one or more (preferably several) point(s) of the second derivative of each measurement curve, and/or
one or more (preferably several) point(s) of the first derivative of each measurement curve and comprise one or more (preferably several) point(s) of the second derivative of each measurement curve, and/or
one or more point(s) of each measurement curve, and/or
one or more percentile(s) of each measurement curve.

The means arranged and/or programmed for the determination preferably comprise means arranged and/or programmed for determination by a random forest method. The device according to the invention can comprise means arranged and/or programmed for learning comprising:
means arranged and/or programmed for acquisition of different DNA melting curves, called reference curves, starting from different reference biological samples belonging to different known initial groups determined before learning, then
means arranged and/or programmed for determination of the descriptors from the reference curves, then
means arranged and/or programmed for construction of a forest by the random forest method, comprising means arranged and/or programmed for construction of several trees by the random forest method, each tree comprising, at each node, the use of one of the descriptors associated with a threshold and making it possible to separate the reference curves into two subsets, each leaf of each tree only corresponding to a single group among the different possible groups.

The means arranged and/or programmed for determining the descriptors can comprise:
means arranged and/or programmed for a preliminary determination of descriptors,
means arranged and/or programmed for, after the preliminary determination, elimination of certain redundant descriptors.

The means arranged and/or programmed for elimination of certain descriptors can comprise means arranged and/or programmed for, for each set of descriptors displaying in pairs a Pearson correlation coefficient greater than 0.95, retention of a single descriptor.

The device according to the invention can comprise:
means arranged and/or programmed for, after acquisition of different reference curves (and/or preferably before construction of the trees), identification of several reference curves corresponding to the same initial group, called ambiguous group, and having profiles separated into several subgroups, and
means arranged and/or programmed for separation of this ambiguous group into several possible groups.

The device according to the invention can comprise:
means arranged and/or programmed for, after acquisition of different reference curves (and/or preferably after construction of the trees), identification of several reference curves corresponding to several initial groups, called merged groups, and having profiles combined in a single group, and
means arranged and/or programmed for unification of these merged groups into a single possible group.

The device according to the invention can further comprise means arranged and/or programmed for calculating a confidence index of the step of determining that the measurement biological sample belongs to a defined group. The means arranged and/or programmed for calculating the confidence index preferably comprise:
means arranged and/or programmed for calculating a distribution of mean proximities between reference curves belonging to the defined group,
means arranged and/or programmed for calculating a mean proximity of the at least one measurement curve with the reference curves belonging to the defined group, and
means arranged and/or programmed for calculating a level of reference curves belonging to the defined group, and having a mean proximity to the other reference curves belonging to the defined group less than the mean proximity of the at least one measurement curve with the reference curves belonging to the defined group.

The device according to the invention can further comprise means arranged and/or programmed for, after the step of determining that the measurement biological sample belongs to a defined group, a refusal to assign the measurement biological sample to any group whatever, as a function of the value of the confidence index.

### DESCRIPTION OF THE FIGURES

Other advantages and characteristics of the invention will become apparent on reading the detailed description of implementations and embodiments, which are in no way limitative, and the following attached drawings:

FIG. 1 is a flowchart of a preferred embodiment of the method according to the invention,

FIG. 2 illustrates several so-called “reference” melting curves 12, for learning the implementation of the method according to the invention,

FIG. 3 illustrates:
several so-called normalized “measurement” melting curves 13 (“technical” replicates) of a sample of which it is sought to determine that it belongs to a defined group among several possible groups,
the percentiles 14 of these measurement curves
the first derivatives 15 of these measurement curves
the second derivatives 16 of these measurement curves

FIG. 4 illustrates several reference melting curves (technical replicates) of several samples (biological replicates) all belonging to the initial group “Mycobacterium fortuitum”,

FIG. 5 illustrates several reference melting curves (technical replicates) of several samples (biological replicates) belonging to the initial group “M. avium” or “M. szulgai”,

FIG. 6 illustrates an example of a tree of a random forest, generated in a case with three descriptors α, β and δ and with two possible groups (group 1 and group 2); an affirmative answer to the question at each node corresponds to selecting the next branch on the left; a negative answer to the question at each node corresponds to selecting the next branch on the right;

FIG. 7 illustrates graphically the principle of a confidence indicator according to the invention,

FIG. 8 is an ROC curve associated with the use of the confidence index based on the intragroup proximities for discriminating between the observations assigned to the correct group or not, and

FIG. 9 illustrates schematically an embodiment of device 100 according to the invention.

### DETAILED DESCRIPTION

As these embodiments are in no way limitative, variants of the invention can be considered comprising only a selection of characteristics described or illustrated hereinafter, in isolation from the other characteristics described or illustrated (even if this selection is isolated within a phrase containing these other characteristics), if this selection of characteristics is sufficient to confer a technical advantage or to differentiate the invention with respect to the state of the art. This selection comprises at least one, preferably functional, characteristic without structural details, and/or with only a part of the structural details if this part alone is sufficient to confer a technical advantage or to differentiate the invention with respect to the prior art.

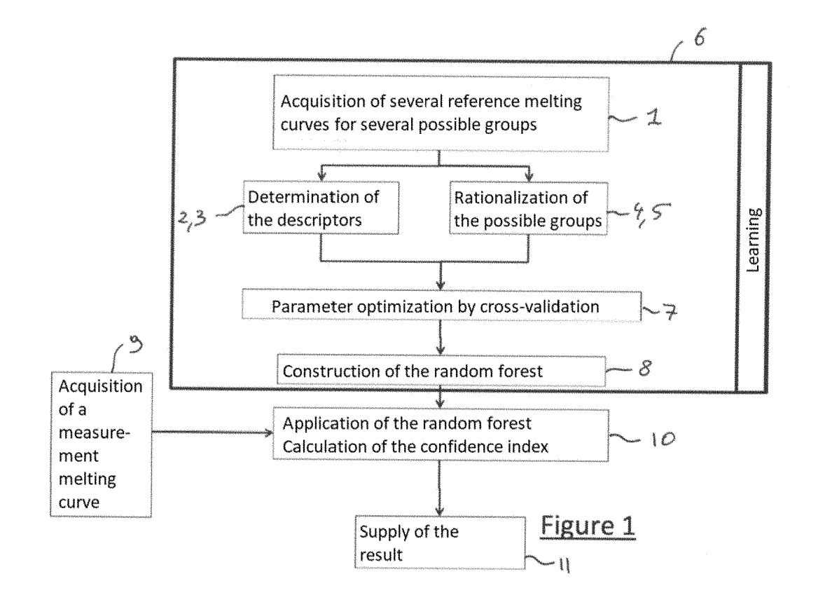

A preferred embodiment of the method according to the invention will therefore be described, with reference to FIGS. 1 to 8. In the remainder of the present description, the expression “method according to the invention” will denote only this embodiment, which is in no way limitative.

The objective of this embodiment is to be able to discriminate between different species. Discrimination between different species of the genus Mycobacterium is selected as an example, which is in no way limitative.

In this embodiment, a “biological sample” corresponds to any type of sample containing, or capable of containing, biological material. Preferably, it is a sample capable of containing mycobacteria and/or a sample capable of containing deoxyribonucleic acid (or “DNA”), or traces of DNA of mycobacteria.

### Principle of Obtaining a Melting Curve

In this embodiment, the molecular biology technique called “high-resolution DNA melting” is also called “HRM” (for “high resolution melting”). This HRM technique is carried out starting from double-stranded DNA. Before analysis by HRM, a fragment of the DNA in which mutations of interest are capable of being located is amplified by PCR (for “polymerase chain reaction”). The sample then contains a large number of copies of the DNA fragment targeted and amplified by the PCR.

The HRM analysis then consists of precise, controlled heating of the DNA fragment amplified by PCR to cause its denaturation. Monitoring the denaturation of the DNA, during the HRM analysis, thus makes it possible to determine a specific melting profile of the target DNA fragment.

The “melting profile” (also called “melting curve”) corresponds to the development of the denaturation of one (or on average of each) DNA molecule as a function of the temperature. Within the meaning of the invention, a melting curve is not necessarily a graphical curve, but can be a list or a table of values of several points of this curve during this denaturation of one (or on average of each) DNA molecule as a function of the temperature.

The PCR reaction comprises for example repetition of the cycle constituted by the following 3 steps:
a step at a temperature from 90° C. to 99° C., for 5 to 30 seconds,
followed by a step at a temperature from 58° C. to 64° C., for 5 to 30 seconds,
followed by a step at a temperature from 70 to 74° C., for 5 seconds to 1 minute.

The three steps constituting the PCR cycle correspond respectively to steps:
of denaturation of the DNA (from 90° C. to 99° C.),
of hybridization of the DNA to the primers (from 58° C. to 64° C.),
and of elongation of the DNA by DNA polymerase starting from the primers (from 70° C. to 74° C.).

The cycle is typically repeated from 40 to 50 times, preferably 45 times.

Said PCR reaction is preferably preceded by a step of initial denaturation of the DNA contained in said biological sample, preferably at 95° C. for 10 minutes.

This step of initial denaturation is a heating step carried out before the PCR cycle. It makes it possible to prepare the DNA of the sample, which will serve as a matrix during the amplification reaction, in particular by completely dehybridizing the double-stranded DNA, by disrupting the secondary structures of the DNA or also by activating the DNA polymerase.

Said PCR reaction is for example carried out using a reaction mixture comprising at least:
the DNA contained in the biological sample
MgCl₂ at a rate of 3 mM,
the primers at a rate of 0.4 μM, and
at least one unit of DNA polymerase.

Said PCR reaction is for example followed by a step of gradual heating between 60° C. and 100° C., preferably from 65° C. to 95° C., to produce denaturation of said amplification product, and obtain a melting profile of said amplification product.

This step of gradual heating corresponds to heating of the sample carried out in a controlled manner, during which the temperature gradually increases in stages over time, such as for example an increase of 0.2° C./second.

The denaturation of said amplification product is typically monitored using a fluorescent marker, preferably selected from LC Green, LC Green Plus, ResoLight, EvaGreen, Chromofy, and SYTO 9.

The steps of amplification and melting were carried out using the high resolution melting kit LightCycler® 480 master kit (Roche). The reaction mixture is composed of 2× Master Mix, MgCl₂, sense and antisense primers, genomic DNA and water, in a final volume of 10 μl. The amplification procedure consists of an initial denaturation followed by 45 cycles of denaturation, hybridization and elongation. After amplification, the melting programme is carried out by heating to 95° C. for 1 minute, cooling to 40° C. for 1 min, followed by the application of a temperature increase from 65 to 95° C. at a rate of increase of 0.2° C./s and continuous measurement of the fluorescence. Each reaction was carried out in triplicate in 96-well plates, with the LightCycler® 480 system (Roche). Each HRM analysis includes a negative control where the DNA matrix has been replaced with water.

It will be noted that, advantageously for the invention, it is easier to obtain a melting curve than to measure the expression of a subset of genes.

### Learning

As illustrated in FIG. 1, the embodiment of the method according to the invention comprises learning 6 comprising:
acquisition 1 of different DNA melting curves, called reference curves, starting from different reference biological samples belonging to different known “initial groups” determined before learning, then
determination 2, 3 of descriptors from the reference curves, then
construction 8 of a forest by the random forest method, comprising construction of several trees by the random forest method, each tree comprising, at each node, the use of one of the descriptors associated with a threshold and making it possible to separate the reference curves into two subsets, each leaf of each tree only corresponding to a single group among the different possible groups. In a classification tree, a leaf always corresponds to a single group, which is generally the majority group of the observations that “fall” into this leaf. The characteristic of the pure leaves is that all the observations of the reference sample that “fall” into this leaf belong to the same group.

The “possible groups”, among which it will then be sought to classify an unknown biological sample, consist of the different “initial groups” of the different reference biological samples used during the learning step, optionally modified (for example by at least one step of separation of groups and/or at least one step of unification of groups as described hereinafter for rationalization of the groups). Preferably, the “possible groups” comprise at least a part of the different “initial groups”.

This learning phase is carried out once for each type of application (with optional possibility of repetition of this phase for inclusion of new reference samples and/or new groups). It has the objective of defining the possible (final) groups and of constructing the decision rule, with:
as inputs of the learning step: library of normalized learning melting profiles with assignment, for each one, to a predefined initial group. Each initial group must be represented by several biological replicates. Each biological replicate must be represented by several technical replicates.
as outputs of the learning step: decision rule for assignment

Importing the normalized signal: for the acquisition step 1 of the different “reference” DNA melting curves serving for learning, the protocol for obtaining melting curves described above is used, and by applying a method of normalization as offered for example by the software associated with the LightCycler® 480 (Roche), 6 series of experiments were carried out on different dates allowing production of 417 HRM profiles (i.e. 417 reference melting curves) corresponding to 19 different species (or “initial groups”) of Mycobacterium. Each species is represented by several technical replicates of several biological replicates (2 to 20 biological replicates per species). “Biological replicates” is the term used for the different biological samples originating from different individuals of one and the same species. “Technical replicates” of one and the same biological replicate is the term used for the different melting curves obtained from the same biological sample. The software input is a text file containing the coordinates of the melting profiles after normalization by the software at the machine output.

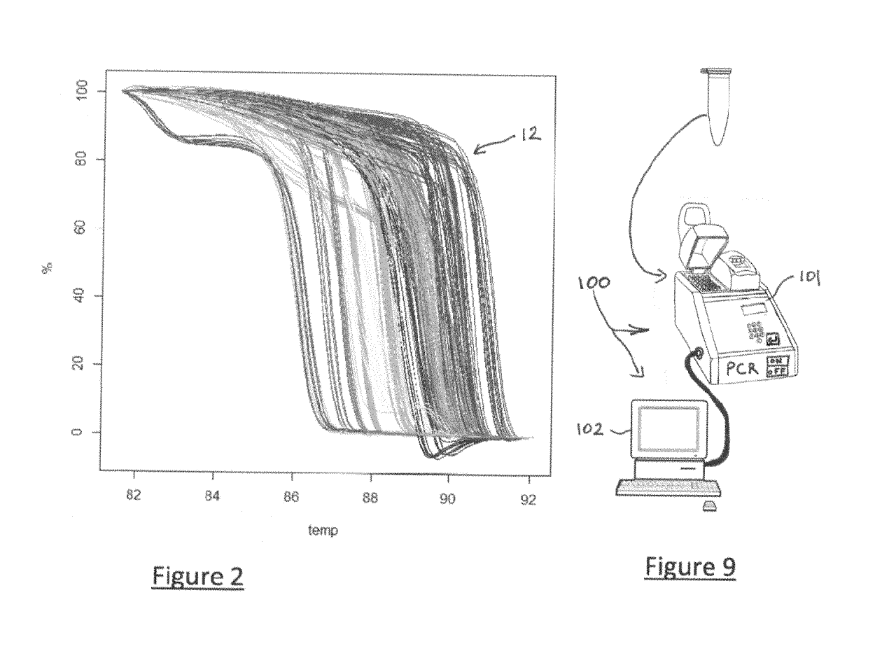

The distribution of the biological replicates among the species is given in Table 1 and the representation of the normalized reference curves 12 associated with the set of technical replicates for the different biological replicates is given in FIG. 2.

TABLE 1

| Species | Number of biological replicates |
|---|---|
| “M. abscessus” | 3 |
| “M. avium” | 20 |
| “M. bohemicum” | 4 |
| “M. bovis” | 4 |
| “M. chelonae” | 4 |
| “M. flavescens” | 3 |
| “M. fortuitum” | 5 |
| “M. gastri” | 4 |
| “M. gordonae” | 5 |
| “M. haemophilum” | 3 |
| “M. immunogenicum” | 3 |
| “M. kansasii” | 9 |
| “M. marinum” | 4 |
| “M. phlei” | 4 |
| “M. phocaicum” | 4 |
| “M. pseudoschottsii” | 3 |
| “M. smegmatis” | 6 |
| “M. szulgai” | 4 |
| “M. wolinskyi” | 3 |
| TOTAL | 95 |

These 19 species form the 19 initial groups.

Determination of the descriptors: the descriptors are then determined. Determination 2, 3 of the descriptors comprises firstly a preliminary determination 2 of the descriptors from the “reference” melting curves D(T) (Denaturation “D” of the DNA (typically in % or as fluorescence signal) as a function of the temperature “T”), which takes into account:
the measurement points of each normalized melting curve (180 points in the present case). Thus 180 descriptors are obtained, which make it possible to characterize each melting curve.
the percentiles (101 quantiles, from 0 to 100): the i^{th} percentile is the term used for the temperature of a normalized melting curve at which i % of the denaturation of the DNA has taken place. Thus 101 descriptors are obtained (obtained by interpolation between the measurement points) corresponding to the 101 percentiles, and which make it possible to characterize each melting curve.

Moreover, for the preliminary determination 2 of the descriptors, the melting curves in the strict sense are supplemented with derived data making it possible to describe the curves to be described more precisely:
the first numerical derivative of each normalized melting curve

$$\frac{dD ( T )}{dT} .$$

Thus, for a melting curve established initially on 180 points (i.e. 180 values of levels of denaturation), 178 additional descriptors are obtained, which make it possible to characterize each melting curve first derivative.
the second numerical derivative of each normalized melting curve

$$\frac{{d}^{ 2} D ( T )}{{dT}^{ 2}} .$$

Thus, for a melting curve established initially on 180 points (i.e. 180 values of levels of denaturation), 176 additional descriptors are obtained, which make it possible to characterize each melting curve second derivative.

Finally, the following are obtained:

180+101+178+176=635 descriptors for describing each melting curve or technical replicate.

Determination 2, 3 of the descriptors comprises:
the preliminary determination 2 of descriptors described above, then
an elimination 3 of certain redundant descriptors.

Redundancy of the data is detrimental to learning the possible groups. Now, there are very strong correlations between successive values on a melting curve or on its derivatives. That is why only one descriptor is retained per set of descriptors displaying in pairs a Pearson correlation coefficient greater than 0.95. Thus, the elimination 3 of certain descriptors comprises, for each set of descriptors displaying in pairs a Pearson correlation coefficient greater than 0.95, retention of a single descriptor. Finally, 208 descriptors are retained (among the initial 635) after elimination of the redundant descriptors, including:
38 points of melting curves
12 percentiles
59 points of the first derivative
99 points of the second derivative.

The location of each descriptor selected is given by vertical lines in FIG. 3.

This clearly illustrates the advantage of the method according to the invention: it can in fact be seen that the derivatives (first and second, in particular second) of the melting curves are very rich in discriminating data making it possible to determine that a biological sample belongs to a given possible group, as they comprise a great part of the descriptors finally retained. This is reflected in finer discrimination between the melting profiles.

Rationalization of the Groups:

The embodiment of the method according to the invention can be applied to a large number of problems or applications with varying learning complexity. It can be required to discriminate between groups that are more or less genetically close. It is therefore impossible, a priori, to know whether all the initial groups can be differentiated by their melting curves. That is why a step of “rationalization of the groups” is inserted during learning. It makes it possible to define the perimeter of the differentiable or non-differentiable initial groups. This step is the result of two main findings:
certain initial groups are constituted by heterogeneous subgroups;
certain initial groups are not differentiable from one another by their melting curves.

In FIG. 2, it can be seen that:

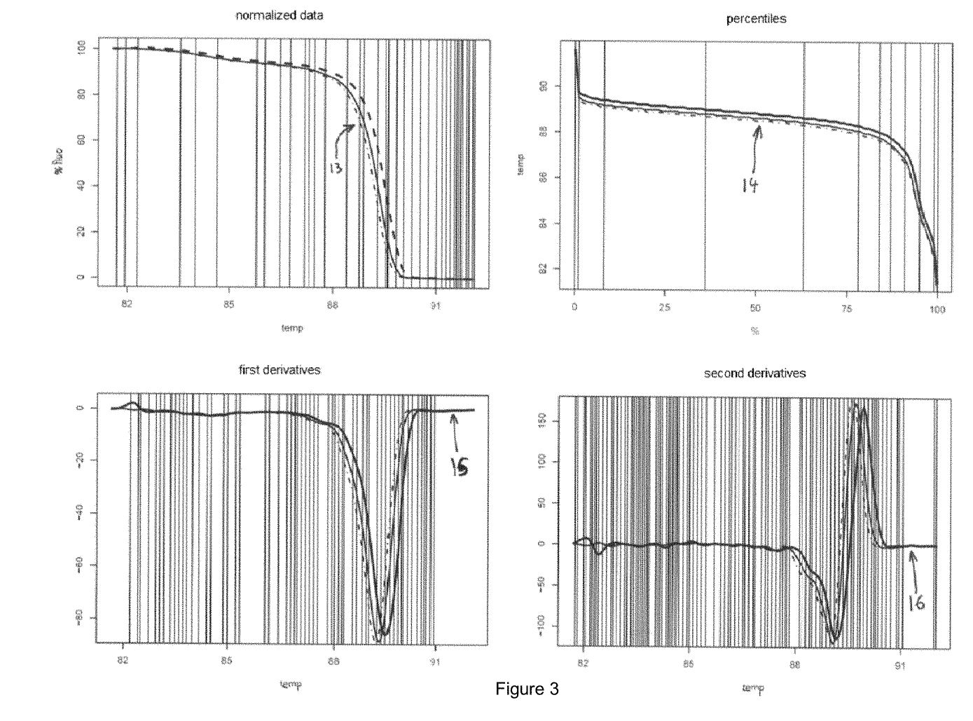

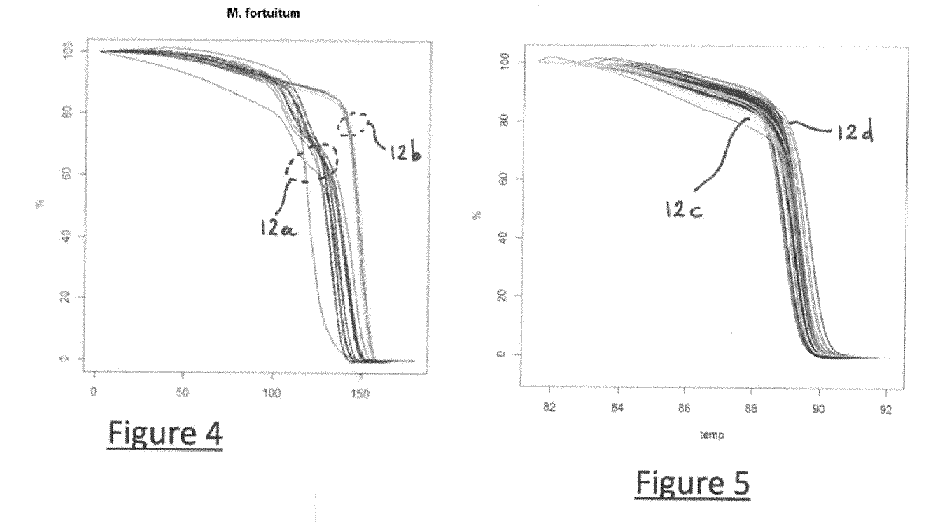

1) the biological or technical replicates can have very different profiles within one and the same initial group. This phenomenon appears in two of the initial groups, in particular the initial group “M. fortuitum” illustrated in FIG. 4. Thus, each of these two initial groups is therefore separated into several (two in the present case) possible subgroups for learning, which makes it possible to facilitate the work of learning; and

2) all the biological or technical replicates of different initial groups can be sufficiently compact.

Thus, in case 1) above, the embodiment of the method according to the invention (more precisely the learning 6) comprises:
after acquisition of different reference curves (but preferably before construction of the trees), identification of several reference curves corresponding to the same initial group, called ambiguous group (such as for example the initial group “M. fortuitum” (FIG. 4), the initial group “M. kansasii”), and having profiles separated into several subgroups; this identification is for example implemented using a criterion of intragroup distance, for which a threshold is defined, and
separation 4 of this ambiguous group into several possible groups 12a and 12b.

Similarly, in case 2) above, the embodiment of the method according to the invention (more precisely the learning 6) comprises:
after acquisition of different reference curves (but preferably after construction of the trees; melting or unification of the groups may require a first construction of the trees), identification of several reference curves corresponding to several initial groups, called merged groups, having profiles combined in a single group; this identification is for example implemented using learning error rates: if errors are made on one group, poorly classified observations can be combined with the observations for which they are the closest, to form a fused or unified group, and
unification 5 of these merged groups into a single possible group;

In this embodiment of the method according to the invention, the initial groups “M. szulgai” 12c and “M. avium” 12d are for example very close, but are not finally unified despite their proximity, owing to the great fineness of analysis of the method according to the invention.

Finally, the following 21 final possible groups listed in Table 2 are obtained:

TABLE 2
Name of each of the possible groups finally
used in the random forest method

“M. abscessus”
“M. avium”
“M. bohemicum”
“M. bovis”
“M. chelonae”
“M. flavescens”
“M. fortuitum 1”
“M. fortuitum 2”
“M. gastri”
“M. gordonae”
“M. haemophilum”
“M. immunogenicum”
“M. kansasii 1”
“M. kansasii 2”
“M. marinum”
“M. phlei”
“M. phocaicum”
“M. pseudoschottsi”
“M. smegmatis”
“M. szulgai”
“M. wolinskyi”

The step of “rationalization of the groups” can, moreover, be iterative, after construction of the random forest described below. Firstly, after optimization of the parameters, the random forest method adapted in cross-validation is applied in two blocks. Then the biological replicates that are assigned to the bad group are given a reference. For each of these replicates, a new group is created, collecting together this badly assigned replicate and the closest biological replicate of the wrongly assigned group. A “hybrid” group has therefore been created, comprising a double tag. The procedure begins again, until all the biological replicates of the learning sample are correctly assigned. At the end of this step a certain number of groups having one or more “tags” is obtained.

Of course, this step can comprise the creation of hybrid groups comprising several initial groups. However, it is very valuable in a context of prediction with a large number of groups, to have the possibility of greatly reducing the number of possibilities. The more so because with this method the whole of the group is not forced to fuse with another, but reasoning is carried out at the scale of the biological replicate. Thus, if an initial group is heterogeneous with a subset of biological replicates that approaches another group, two possible final groups will finally be obtained: a final group only comprising replicates of the initial group and a hybrid final group.

Definition of a Method for Predicting and Determining the Parameters of the Learning Method:

The learning 6 finally comprises the construction 8 of the forest by the random forest method.

The operation of this random forest method is adapted here to the structure of the data as technical replicates/biological replicates according to the invention. The technical replicates make it possible to take into account the technical variability of obtaining the melting profiles (variability that is quite limited). The biological variability is at the heart of the learning, as it reflects the variability with which the embodiment of the method according to the invention will be faced under real conditions of use. It is linked to the sequence differences that can be observed between individuals of one and the same possible group.

To discriminate between the k different possible groups (k=21 possible groups in this example, cf. Table 2), the well-known random forest method is therefore used (cf. references [2], [3], [4] for further details concerning the well-known general considerations of this random forest method). The principle of this method, based on classification trees, is to construct several classification trees using for each tree a subset of the n starting reference melting curves (also called “observations”) (n=417 reference melting curves in this example) and, for each node of the tree, a subset of the p starting descriptors (also called “variables”) (p=208 descriptors in this example). This method depends on two parameters:
ntree the number of trees constructed, i.e. the total number of trees involved in the final decision rule and
mtry the number of descriptors studied at each node of each tree, with mtry≤p, i.e. the number of descriptors randomly selected from the set of the descriptors available during learning of each node of each tree.

These two parameters are determined during an optimization step 7 by cross-validation in two blocks on the learning data (reference curves). For this step 7 (which forms part of the learning 6), and for each use of cross-validation, the work is carried out at the scale of the biological replicate, i.e. at each step of the cross-validation, the technical replicates of a biological replicate are either all assigned to the learning block, or all assigned to the validation block. This constraint has the advantage of most closely mimicking actual learning conditions. The parameters selected are those that maximize the mean percentages of “well classifieds” obtained on 100 random distributions in learning/test blocks. Thus, for each possible value of the pair (ntree, mtry), a forest is constructed (with several trees) by the random forest method on the basis of half of the n observations, then this forest is tested on the other half of the n observations for which it is in reality already known whether or not it belongs to each of the k possible groups; then the value of the pair (ntree, mtry) is selected, having constructed the forests that give the best results on average (since 100 random distributions are carried out, including 100 forests, for each pair of values). An optimum number of ntree=1000 trees and of mtry=10 variables per node is obtained.

Then the ntree=1000 trees of the random forest are constructed using the n=417 observations.

For constructing each tree:
100 observations (or reference curves) are taken at random from the n=417 available, and
at each node 17 of each tree, mtry=10 descriptors are taken at random from the p=208 available, and from the mtry=10 descriptors taken, the descriptor and the associated threshold are selected which together optimize the Gini index; there are as many nodes as necessary for each branch end 18 or “leaf” 18 to be pure, i.e. the observations that “fall” there all belong to the same initial group.

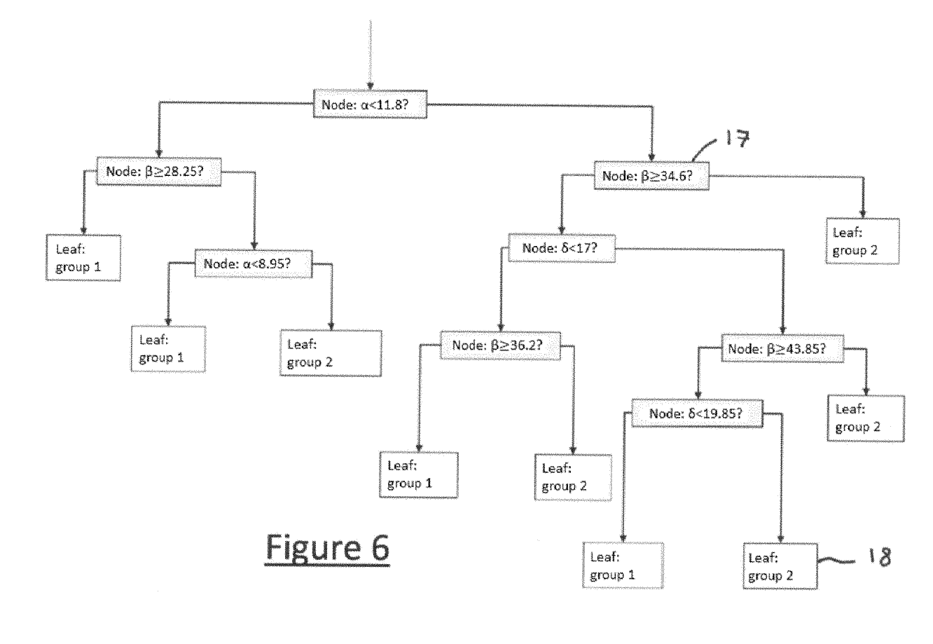

FIG. 6 illustrates an example of a tree of a random forest, in a case with three descriptors α, β and δ (p=3) and with two possible groups (k=2, group 1 and group 2). This tree, even if it does not correspond to the case of the embodiment of the method according to the invention in FIG. 2 (k=21 possible groups, p=208 descriptors, with trees much too large to be illustrated) illustrates perfectly the principle of the trees constructed in this embodiment of the method according to the invention.

Each node 17 corresponds to a question posed relating to a descriptor, typically: does this descriptor have a value below (or less than or equal to) a threshold?

For example:
For each of the descriptors forming part of the points of melting curves (38 descriptors): does the percentage denaturation (or other arbitrary unit of the melting curve), for a temperature value specific to this descriptor, have a value below (or less than or equal to) a given threshold?
For each of the descriptors forming part of the percentiles (12 descriptors): does the temperature, for a value of percentage denaturation (or other arbitrary unit of the melting curve) specific to this descriptor, have a value below (or less than or equal to) a given threshold?
For each of the descriptors forming part of the points of the first derivative (59 descriptors): does the first derivative of the melting curve, for a temperature value specific to this descriptor, have a value below (or less than or equal to) a given threshold?
For each of the descriptors forming part of the points of the second derivative (99 descriptors): does the second derivative of the melting curve, for a temperature value specific to this descriptor, have a value below (or less than or equal to) a given threshold?

Each leaf 18 corresponds to one of the k possible final groups.

Learning the Confidence Index:

By construction, the random forest method makes it possible to calculate proximities between observations by studying the number of trees in which two observations “fall” in the same leaf. This proximity is used for calculating a confidence indicator of the prediction and therefore for optionally refusing to assign an observation to one of the possible groups.

Thus, after construction of the random forest, during the learning step 6, the distribution of the proximities in pairs is calculated for all the pairs of biological replicates of the learning library belonging to one and the same possible group. The proximities between biological replicates are defined by the minimum value of the proximities calculated between its technical replicates (so-called complete linkage method). This distribution can be smoothed by a kernel method. This operation is repeated for each possible group, thus obtaining a distribution of the intragroup distances specific to each group.

### Prediction: Classification of an Unknown, So-Called “Measurement” Biological Sample

Prediction is the current step of the embodiment of the method according to the invention. It has the objective of applying the decision rule to a biological sample in order to obtain assignment to one of the possible final groups (also called “classes”) obtained as the output of the learning (in particular after the step of rationalization of the groups), this assignment being matched with a confidence indicator. This therefore results in:
as inputs of the prediction step: a set of melting profiles (technical replicates) obtained from one and the same “measurement” biological sample.
as outputs of the prediction step: decision on assignment of the “measurement” sample with confidence indicator, or optionally refusal of assignment to any one of the possible groups.

In fact, the objective of the embodiment of the method according to the invention is then, based on the description of “measurement” samples by their melting curve, to decide whether or not to assign this individual to one of the k possible final groups defined during learning (supervised method) and to assign a confidence indicator to the proposed decision.

Thus, the embodiment of the method according to the invention comprises acquisition (9) of at least one normalized curve (as seen above) of DNA melting of the measurement biological sample, called at least one measurement curve, each measurement curve comprising different points, each point corresponding to a quantity that is proportional to (for example a fluorescence signal) or representative of a level (typically in %) or of a quantity of denaturation of the DNA of the measurement sample as a function of a temperature; this acquisition can comprise carrying out the PCR and the melting curve itself (in the laboratory), and/or a simple downloading of data (computer data for example) of this melting curve.

Optionally, the PCR for this melting curve can be carried out in the simultaneous presence of several primer pairs targeting several target DNA molecules. We then refer to “multiplexing” conditions.

The embodiment of the method according to the invention further comprises determination 10, by the random forest method based on the forest of trees constructed during the learning phase, that the measurement biological sample belong to a defined group among k different possible final groups. This determination comprises an analysis, by the random forest method based on the forest of trees constructed during the learning phase, of descriptors originating from the at least one measurement curve, the descriptors comprising:
one or more points of each measurement curve and/or
some or all of the 101 percentiles of the levels of denaturation of each measurement curve, and/or
one or more points (typically at least 30 points) of the first derivative of each measurement curve and/or
one or more points (typically at least 30 points) of the second derivative of each measurement curve.

The technical replicates of the measurement biological sample are subjected independently to the random forest and a possible group is assigned to each of them. By default, the measurement biological sample is assigned to the majority group among the groups predicted for each technical replicate. In the case of multiple groups, the confidence index can be used for deciding.

As random forests are stochastic methods (several applications can give different results), this method is applied several times (3 times in this embodiment) for predicting the assignment of the biological sample.

The location of each descriptor is given by vertical lines in FIG. 3.

FIG. 3 illustrates:
at top left, three measurement melting curves 13 (three technical replicates) of one and the same measurement sample of which it is sought to determine that it belongs to one of the k possible groups;
at top right, three curves 14 illustrating the percentiles corresponding to the three technical replicates 13;
at bottom left, the three first derivatives 15 of the three measurement melting curves 13, and
at bottom right, the three second derivatives 16 of the three measurement melting curves 13.

### Calculation of a Confidence Indicator:

The quality of the embodiment of the method according to the invention is determined by the quality of the initial learning library. The richer this is in biological variability, the more the learning will be accurate and generalizable to a great diversity of new samples.

However, regardless of the quality of the learning library, during the prediction of new samples, it is still possible to encounter samples that are totally foreign to it. In that case the conventional learning methods will nevertheless supply a prediction by assigning the new sample to the possible group it is closest to. The embodiment of the method according to the invention must be capable of refusing to assign a new sample to any possible group whatever.

For this, the embodiment of the method according to the invention comprises calculation of a confidence index of the step of determining that the measurement biological sample belongs to a defined group.

This step of calculating a confidence indicator has a dual objective:
to quantify the reliability of the predictions made;
to allow a biological replicate to predict not being assigned to any possible group.

Random forests have the advantage of supplying measurements of proximity between observations. For further details about this well-known concept of “proximity” in the random forest method, reference can be made for example to references [3] and [4].

These measurements are used for supplying the confidence index. In fact, if the observation to be predicted is close to the observations of the possible group to which it is assigned then the quality of the classification is potentially better than if the observation to be predicted is far from the observations of the possible group to which it is assigned. This principle was used for constructing the confidence index.

The learning data were taken as a basis for calculating the distribution of the mean proximities of the reference melting curves of one and the same possible group. Then, when a measurement biological sample is assigned to a possible group, its mean proximity to the biological replicates of this group is calculated and is compared to the proximities of the reference melting curves of this possible group. It is then possible to calculate the percentage of reference melting curves the proximity of which is less than that of the measurement melting curve to be predicted. This percentage is an estimate of the probability of belonging to the predicted group and is used as the confidence index.

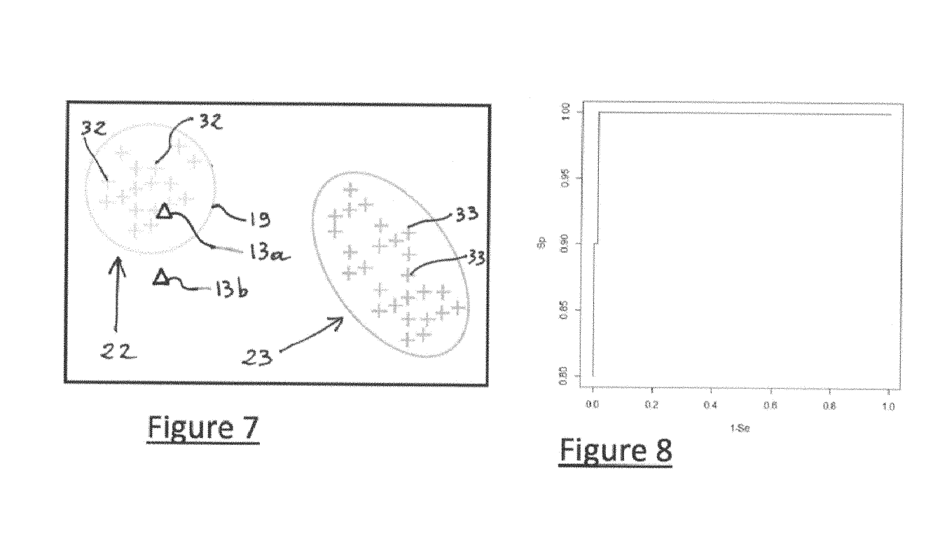

FIG. 7 illustrates the principle of this index with two possible groups 22, 23 to which reference melting curves (crosses), 32 and 33 respectively, belong.

In the case where the (or each of the or the mean of the) measurement curve(s) 13a (triangle):
is closer to group 22 than to group 23 and
has its proximity to the melting curves 32 of group 22 consistent with the proximities that are observed between the melting curves 32 of group 22;

then the sample corresponding to the curves 13a is considered to belong to group 22, and the embodiment of the method according to the invention confirms that the group determined is indeed group 22.

In the case where the (or each of the or the mean of the) measurement curve(s) 13b (triangle):
is closer to group 22 than to group 23, but
has its proximity to the melting curves 32 of group 22 far less than the proximities that are observed between melting curves 32 of group 22,

then the sample corresponding to curves 13b belongs neither to group 22 nor to group 23, and preferably the embodiment of the method according to the invention comprises a refusal to assign the measurement biological sample to the defined group 22 and even optionally to any group whatever.

During the learning phase, the distribution of the proximities in pairs of all the pairs of biological replicates of the learning library belonging to one and the same group was calculated.

During the prediction step, for any new observation (i.e. for any new “measurement” melting curve), its mean proximity to the reference biological replicates of the group to which it was assigned is calculated by the same method. The overall distribution obtained previously is then used for calculating the estimate of the probability of belonging to this group.

If the new observation passes this step, the probability of belonging to this possible group is supplied to the user at the same time as the predicted group.

The possibility of applying this last step is of course dependent on sufficient size of the learning library.

FIG. 8 shows the ROC (“Receiver Operating Characteristic”) curve associated with this index (AUC=0.9988, AUC being the area under the ROC curve or “area under the curve”).

This FIG. 8 is of excellent quality. In fact, referring to FIG. 7, if a threshold value equal to 0.14 is used for the confidence index, then all the observations having an index below this threshold (symbolized by the loop 19) are poorly classified observations while only one well-classified observation has an index above this threshold. Owing to this index, the well-classified observations can therefore be identified very satisfactorily.

In the case of assignments to several possible groups (as a result of the different applications of the random forests or contradictory results of the different technical replicates) or a low confidence index, the proximity to the set of the possible groups predicted at least once (out of all of the trees of the forest) for a biological replicate can be calculated again. If one of these possible groups displays an index above the threshold of 0.14, this measurement curve can be tagged as probably belonging to the possible group having the maximum index value.

Applying this rule to the reference curves to test its efficacy, melting curves are “recovered” that had mostly been assigned to the wrong species but for which the correct species had been predicted at least once and with which the calculated confidence index is greater than 0.14.

Finally, the confidence index can be used for deciding between two possible groups that would have been assigned the same number of times to a measurement melting curve.

Thus, to summarize, calculation of the confidence index comprises:
calculating a distribution of mean proximities between reference curves belonging to the defined group,
calculating a mean proximity of the measurement biological sample with the reference curves belonging to the defined group, and
calculating a level of reference curves belonging to the defined group, and having a mean proximity to the other reference curves belonging to the defined group less than the mean proximity of the at least one measurement curve with the reference curves belonging to the defined group.

After the step of determining that the measurement biological sample belongs to a defined group, the implementation of the method according to the invention comprises (as step 11 supplying the result, typically displayed on a screen or stored in a computer memory or electronic memory) a refusal or not to assign the measurement biological sample to any possible group whatever as a function of the value of the confidence index, more exactly:
a refusal to assign the measurement biological sample to any possible group whatever if the confidence index is below a threshold value, or
an acceptance to assign the measurement biological sample to one of the possible final groups if the confidence index is above a threshold value

By combining the raw results of the random forest and the use of the confidence index, 95.74% of the observations are correctly assigned. As for the remaining 4.26% of observations, they are clearly identified by the embodiment of the method according to the invention as being suspect with respect to their assignment.

In the case of the curves in FIG. 3, the sample is identified as belonging to the group “abscessus”.

In this embodiment of the method according to the invention, each of the following steps:
acquisition 1
determination of the descriptors 2,3
rationalization of the groups 4, 5
learning 6
optimization of the parameters 7
construction of the random forest 8
acquisition 9
step 10 of analysis of the descriptors, application of the random forest, determination of the defined group among the several possible groups, calculation of the confidence index and decision to confirm assignment of the measurement biological sample to the defined group or refusal to assign the measurement biological sample to any group whatever,
supply or display of the result 11
   is not carried out purely abstractly or purely intellectually but involves the use of a technical means (just like generally all the other steps of this embodiment of the method according to the invention).

FIG. 9 illustrates schematically an embodiment of device 100 according to the invention arranged and programmed for implementing this embodiment of the method according to the invention.

The device 100 comprises means 102 arranged for and programmed for implementing each of the following steps:
determination of the descriptors 2,3
rationalization of the groups 4, 5
optimization of the parameters 7
construction of the random forest 8
step 10 of analysis of the descriptors, application of the random forest, determination of the defined group among the several possible groups, calculation of the confidence index and decision to confirm assignment of the measurement biological sample to the defined group or refusal to assign the measurement biological sample to any group whatever
supply or display of the result 11.

The device 100 comprises means 101 and 102 arranged for and/or programmed for implementing:
learning step 6 and more precisely step 1 of acquisition of the reference curves,
step 9 of acquisition of at least one measurement curve.

The means 102 comprise a computer, and/or a central or calculating unit, and/or an analogue electronic circuit (preferably dedicated), and/or a digital electronic circuit (preferably dedicated), and/or a microprocessor (preferably dedicated), and/or software. These means 102 preferably further comprise a screen or printing means or means for exporting data for step 11 of supply or display of the result.

The means 101 comprise a PCR machine, and/or according to the variant can comprise computing means (software combined with a USB port, an SD card reader, a connection to a computer network, etc.) arranged and programmed for loading and reading the DNA melting curves. Thus, these means 101 are connected to or form part of the means 102.

Of course, the invention is not limited to the examples which have just been described and numerous adjustments can be made to these examples without exceeding the scope of the invention.

For example, another proof of the concept was carried out on other microorganisms including Coxiella burnetii, Chiamydophila spp, Neospora caninum, Toxoplasma gondii, and Anaplasma with the same success. Experiments have demonstrated that the method developed made it possible to identify the different pathogens, including under multiplexing conditions, i.e. via PCR amplification in the simultaneous presence of all the primer pairs targeting the target DNA molecules of all the (five) pathogens mentioned above.

In general, the invention is applicable to any biological sample, in particular human, animal, plant, viral, bacterial, of Archaea, fungal, yeast, viroid, of a eukaryote, or of a protozoon.

Of course, the different characteristics, forms, variants and embodiments of the invention can be combined with one another.

### REFERENCES CITED

[1]: WO2013/166373
[2]: “Random Forests”, Leo Breiman, published in “Machine learning”, 45, 5-32, 2001.
[3]: “Classification and Regression by RandomForest”, Andy Liaw and Matthew Wiener, R News, Vol. 2/3, December 2002, ISSN 1609-3631.
[4]: “Manual on setting up, using, and understanding Random Forests V3.1”, Leo Breiman, 2002

1. A method for classifying a measurement biological sample, comprising:
acquisition (9) of at least one DNA melting curve of the measurement biological sample, called at least one measurement curve;
determination (10) that the measurement biological sample belongs to a defined group among different possible groups, by analysis of descriptors originating from the at least one measurement curve,
characterized in that the descriptors comprise one or more points of the second derivative of each measurement curve.

2. The method according to claim 1, characterized in that the descriptors comprise one or more points of the first derivative of each measurement curve.

3. The method according to claim 1 or 2, characterized in that the descriptors comprise one or more points of each measurement curve.

4. The method according to any one of the preceding claims, characterized in that the descriptors comprise one or more percentiles of each measurement curve.

5. The method according to any one of the preceding claims, characterized in that the determination (10) comprises determination by a random forest method.

6. The method according to claim 5, characterized in that it comprises learning (6) comprising:
acquisition (1) of different DNA melting curves, called reference curves, starting from different reference biological samples belonging to different initial groups; then
determination (2, 3) of the descriptors starting from the reference curves; and then
construction (8) of a forest by the random forest method, comprising construction of several trees by the random forest method, the variable or variables studied at each node of each tree comprising one or more of the descriptors, each leaf of each tree only corresponding to a single group among the different possible groups.

7. The method according to claim 6, characterized in that determination (2, 3) of the descriptors comprises:
a preliminary determination (2) of descriptors; and then
an elimination (3) of certain redundant descriptors.

8. The method according to claim 7, characterized in that the elimination (3) of certain descriptors comprises, for each set of descriptors displaying in pairs a Pearson correlation coefficient greater than 0.95, retention of a single descriptor.

9. The method according to any one of the preceding claims, characterized in that it comprises:
after acquisition of different reference curves, identification of several reference curves corresponding to the same initial group, called ambiguous group, and having profiles separated into several subgroups; and
separation (4) of this ambiguous group into several possible groups.

10. The method according to any one of the preceding claims, characterized in that it comprises:
after acquisition of different reference curves, identification of several reference curves corresponding to several initial groups, called merged groups, and having profiles combined in a single group; and
unification (5) of these merged groups into a single possible group.

11. The method according to any one of the preceding claims, characterized in that it further comprises calculation of a confidence index of the step of determining that the measurement biological sample belongs to a defined group.

12. The method according to claim 11, characterized in that calculation of the confidence index comprises:
calculation of a distribution of mean proximities between reference curves belonging to the defined group;
calculation of a mean proximity of the at least one measurement curve with the reference curves belonging to the defined group; and
calculation of a level of reference curves belonging to the defined group, and having a mean proximity to the other reference curves belonging to the defined group less than the mean proximity of the at least one measurement curve with the reference curves belonging to the defined group.

13. The method according to claim 11 or 12, characterized in that it further comprises, after the step of determining that the measurement biological sample belongs to a defined group, a refusal to assign the measurement biological sample to any group whatever, as a function of the value of the confidence index.

14. The method according to any one of the preceding claims, characterized in that the acquisition of at least one DNA melting curve of the measurement biological sample comprises acquisition of at least one melting curve of a result of a PCR obtained in the simultaneous presence of several primer pairs targeting several target DNA molecules.

15. A device (100) for classifying a measurement biological sample, comprising:
means (101) arranged and/or programmed for acquisition (9) of at least one DNA melting curve of the measurement biological sample, called at least one measurement curve;
means (102) programmed for determining (10) that the measurement biological sample belongs to a defined group among different possible groups, by analysis of descriptors originating from the at least one measurement curve;
characterized in that the descriptors comprise one or more points of the second derivative of each measurement curve.

16. The device according to claim 15, characterized in that the descriptors comprise one or more points of the first derivative of each measurement curve.

17. The device according to claim 15 or 16, characterized in that the descriptors comprise one or more points of each measurement curve.

18. The device according to any one of claims 15 to 17, characterized in that the descriptors comprise one or more percentiles of each measurement curve.

19. The device according to any one of claims 15 to 18, characterized in that the means (102) programmed for determination (10) comprise means (102) programmed for determination (10) by a random forest method.

20. The device according to claim 19, characterized in that it comprises means (101, 102) arranged and/or programmed for learning (6) comprising:
means (101) arranged and/or programmed for acquisition (1) of different DNA melting curves, called reference curves, starting from different reference biological samples belonging to different initial groups; then
means (102) arranged and/or programmed for determination (2, 3) of the descriptors from the reference curves; then
means (102) arranged and/or programmed for construction (8) of a forest by the random forest method, comprising means (102) arranged and/or programmed for construction of several trees by the random forest method, the variable or variables studied at each node of each tree comprising one or more of the descriptors, each leaf of each tree only corresponding to a single group among the different possible groups.

21. The device according to claim 20, characterized in that the means (102) programmed for determination (2, 3) of the descriptors comprise:
means (102) programmed for the preliminary determination (2) of descriptors,
means (102) programmed for, after the preliminary determination, an elimination (3) of certain redundant descriptors.

22. The device according to claim 21, characterized in that the means (102) programmed for the elimination (3) of certain descriptors comprise means (102) programmed for, for each set of descriptors displaying in pairs a Pearson correlation coefficient greater than 0.95, retention of a single descriptor.

23. The device according to any one of claims 15 to 22, characterized in that it comprises:
means (102) programmed for, after acquisition of different reference curves, identification of several reference curves corresponding to the same initial group, called ambiguous group, and having profiles separated into several subgroups; and
means (102) programmed for separation (4) of this ambiguous group into several possible groups.

24. The device according to any one of claims 15 to 23, characterized in that it comprises:
means (102) programmed for, after acquisition of different reference curves, identification of several reference curves corresponding to several initial groups, called merged groups, and having profiles combined in a single group; and
means (102) programmed for unification (5) of these merged groups into a single possible group.

25. The device according to any one of claims 15 to 24, characterized in that it further comprises means (102) programmed for calculating a confidence index of the step of determining that the measurement biological sample belongs to a defined group.

26. The device according to claim 25, characterized in that the means (102) programmed for calculating the confidence index comprise:
means (102) programmed for calculating a distribution of mean proximities between reference curves belonging to the defined group;
means (102) programmed for calculating a mean proximity of the at least one measurement curve with the reference curves belonging to the defined group; and
means (102) programmed for calculating a level of reference curves belonging to the defined group, and having a mean proximity to the other reference curves belonging to the defined group less than the mean proximity of the at least one measurement curve with the reference curves belonging to the defined group.

27. The device according to claim 25 or 26, characterized in that it further comprises means (102) programmed for, after the step of determining that the measurement biological sample belongs to a defined group, a refusal to assign the measurement biological sample to any group whatever, as a function of the value of the confidence index.
